(19) United States
(12) Patent Application Publication
(10) Pub. No.: US 2025/0161589 A1
(43) Pub. Date: May 22, 2025

(54) CURVED NEEDLE FOR DERMAL FILLING

(71) Applicant: CIRCULUS BY JULIE HORNE PTY LTD, Melbourne (AU)

(72) Inventor: Erdihan Avjioglu, Melbourne (AU)

(21) Appl. No.: 18/730,354

(22) PCT Filed: Jan. 19, 2023
(86) PCT No.: PCT/AU2023/050024

§ 371 Date: Jul. 19, 2024

(30) Foreign Application Priority Data
Jan. 19, 2022 (AU) 2022900089

(51) Int. Cl. A61M 5/32

(52) U.S. Cl. CPC A61M 5/3286; A61M 2210/04

(57) ABSTRACT

This invention relates to a curved needle for the injection of dermal fillers into the lips and perioral area.

### CROSS-REFERENCE TO RELATED APPLICATION(S)

This application is a Section 371 National Stage Application of International Application No. PCT/AU2023/050024, filed Jan. 19, 2023 and published as WO 2023/137516 A1 on Jul. 27, 2023, in English, and claims priority to Australian Application No. 2022900089, filed Jan. 19, 2022.

### FIELD OF THE INVENTION

The present invention is generally directed toward a hypodermic needle for the injection of dermal fillers.

### BACKGROUND TO THE INVENTION

Dermal fillers (also known as injectable fillers, facial fillers, or cosmetic fillers) are gel-like materials that are injected into the skin to provide facial volume, augment facial features, and fill in facial wrinkles. Commonly used dermal fillers include hyaluronic acids, collagens, and biosynthetic polymers. Dermal fillers are provided in gel form, and are designed to remain in place where injected to fill in features and provide volume as desired.

It is particularly common to use dermal fillers to add volume to the lip and mouth region of the face, including for the purposes of:
restoring lip size noting that lips may get smaller or thinner as a result of aging;
correcting the shape of lips to avoid asymmetry;
smoothing wrinkles around the side of the mouth; and
generally creating a smoother, fuller appearance of the lip and mouth regions.

A dermal filler for use in the lip region is commonly called a lip filler. A typical lip filler procedure may proceed as follows:
prior to injection of dermal/lip filler, a topical anaesthetic will be applied to the lips. The topical anaesthetic will numb the lips to remove or reduce pain and ensure the process is made as comfortable as possible. If the recipient has an allergy to the relevant topical anaesthetic, a nerve block injection may instead be made to numb the lips. About 15 to 30 minutes from application or injection of anaesthetic (as the case may be), the lips should be numb;
dermal filler will then be injected into the relevant parts of the lips, potentially including the edges of the lips (vermillion border), the curve in the centre of the upper lip (Cupid's bow) and the oral commissures (corners of the mouth). On average, around 1 mL of lip filler will be inserted into the lips; and
an ice pack may be applied to the lips throughout and following the procedure to minimize swelling and bruising.

As shown in FIG. 1, skin 1 and tissue layers most relevant to a dermal filler procedure include:
the epidermis 2—being the outermost layer of the skin 1 and forming the waterproof, protective wrap over the body's surface. Among other things, the epidermis 2 provides a barrier to infection and helps the skin 1 regulate body temperature. The epidermis 2 contains no blood vessels and is nourished by diffusion from the dermis. The epidermis 2 can vary in thickness depending upon location, but in the lip-region has a thickness of approximately 60 μm;
the dermis 3—being the layer beneath the epidermis 2 and tightly connected to the epidermis 2 by a basement membrane. The dermis 3 contains nerve endings that provide the skin 1 with a sense of touch and heat, as well as hair follicles, sweat glands, sebaceous glands, apocrine glands, lymphatic vessels and blood vessels. Blood vessels in the dermis 3 provide nourishment and waste removal from its own cells as well as from the stratum basale of the epidermis 2. The dermis 3 can vary in thickness depending upon location, but in the lip region typically has a thickness in the range of 600-1000 μm; and
the hypodermis 4 is also known as sub-cutaneous tissue. Lying below the dermis 3, the hypodermis 4 does not form part of the skin 1. The purpose of the hypodermis 4 is to attach the skin 1 to underlying bone and muscle as well as supplying it with blood vessels and nerves. The hypodermis 4 consists of loose connective tissue, adipose tissue and elastin.

While dermal filler can be injected into various layers depending on intended application, the dermis 3 represents the layer into which dermal filler is most commonly injected. Injecting into the superficial to mid dermis 3 in particular can stop the skin folding process which creates wrinkles, and the dermis 3 also provides the most superficial traction or support, meaning that injection into the dermis 3 can help to correct skin 1 weaknesses and the effects of gravity. The dermis 3 is also generally considered a relatively safe location for injection, as the dermis 3 is not close to significant blood vessels. Generally, when undertaking a lip filler procedure, the dermis 3 is selected as the intended layer for injection of dermal filler.

Dermal filler injections are traditionally made with a short needle, and many commercial fillers are in fact sold in packs comprising two such needles for use. The benefits of a needle are considered to be that:
a needle punctures the skin and as many tissue planes of the face as required to get to the intended layer in which dermal filler is to be deposited;
a needle can be injected into precise locations on the face and is therefore suitable for delicate work.

While the ability of a needle to puncture tissue planes may be seen as an advantage in certain situations, using a needle also presents risks. There is for example a risk that the needle will pierce blood vessels lying below the dermis, causing bruising. More seriously, a needle may inject filler into a blood vessel, leading to a vascular occlusion. Less seriously, but nevertheless not desired, the angle at which a needle is inserted can mean that dermal filler is injected at different depths and within different layers within or below the skin, leading to an uneven cosmetic result whereby some dermal filler is deposited too deep and some too superficial—in turn leading to lumpy, undesirable cosmetic results.

This is exemplified in FIG. 2, which in FIG. 2A shows a needle 5 passing through the epidermis 2, the dermis 3 and the hypodermis 4. As shown in FIG. 2B, injection of dermal filler 6 as the needle 5 passes out of the skin can lead to deposition of dermal filler 6 at various depths including within the hypodermis 4.

With reference to FIG. 3, lips are commonly injected using vertical injections from the vermillion border 9 aimed at the body (wet/dry junction) 14 of the lip. Also shown in FIG. 3 is the submucosa 8, the outer lip 10, the sebaceous gland 11, the muscle layer 12 and the salivary glands 13. As exemplified in FIG. 4, use of a needle 5 according to the prior art may be seen to increase the risk that a needle 5 may puncture tissue planes and lead to dermal filler 6 being deposited at different depths and within different layers within or below the skin 1, leading to uneven cosmetic results.

Use of existing needles can further limit the cosmetic effectiveness of a dermal filler injection procedure as now discuss. As noted above, dermal filler procedures are commonly used to provide facial volume and augment facial features. In providing facial volume, it is generally desirable to provide a rounder fuller shape profile reflecting the rounder shapes found on the human face, as opposed to for example providing flat or square shaped filler profiles. Taking the lips for example, FIG. 13A shows human lips detailing tubercles 23, cupids bow and the vermillion border 9. A common dermal filler process involves filling of the lower left and lower right tubercles 23. In such a process it is desirable to provide rounder and fuller lower tubercles whereby the combined effect generally provides a ‘heart-shape’ across the two lower tubercles 23. To enable this, it is desired to deposit dermal filler in a manner resulting in a generally round/circular bolus region for each lower tubercle 23. Providing a round/circular bolus region for each lower tubercle is difficult to achieve with existing needles, which are generally suited to insertion into and passing along the dermis layer 3 in a straight line. That is, existing needles are suited to injecting dermal filler into the dermis 3 in a straight line whereby a straight line of dermal filler is deposited. While such needles are suitable for providing a square or rectangular bolus, it can be quite difficult to obtain a generally circular bolus out of straight line depositions of dermal filler—as exemplified in FIG. 13B.

FIGS. 13C to 13D further illustrate scenarios in which deposition using existing needles can provide undesirable results. FIG. 13C illustrates deposition of dermal filler along the cupids bow. Depending on facial and skin structure, it can be generally desirable to deposit a line of filler so as to directly follow along the cupid's bow. As illustrated in FIG. 13C, providing a desirable line of filler can be quite difficult with existing needles, since such needles are suited to depositing dermal filler in a straight line. FIG. 13D shows the use of vertical injections into the lower lip, which are commonly performed to ‘fill out’ the lower lip to increase height and definition. Using an existing needle often results in deposition of dermal filler in a straight line, providing the opposite of the desired result whereby instead of providing a fuller, rounder effect—the deposition results in flattening out of the lower lip.

More recently, it is has become common to inject dermal filler using a cannula. In comparison to the needles used in dermal filler procedures, a cannula is longer, thinner, flexible, and comprises a blunt end. To use a cannula a needle is first used to puncture the skin, creating an entry point for the cannula to pass through. Due to its blunt tip and flexible configuration, a cannula is less likely to piece blood vessels. Rather, a cannula is more likely to stay within a particular dermal layer. The length and flexibility of a cannula further mean that a cannula requires less entry points into the skin to deliver dermal filler across the treatment area. However, the length and flexibility of a cannula can create its own disadvantages. A cannula is seen as allowing less control and accuracy in areas that require precision, for example when treating the fine “smoker's lines” around the lips where a light hand is required to deliver tiny amounts of dermal filler quite superficially.

It would be desirable to develop a new product for delivery of dermal fillers which addresses one or more issues with the existing art, or at least provides a viable alternative thereto.

### SUMMARY OF THE INVENTION

According to a first aspect of the invention, there is provided a needle for injecting dermal filler into the dermis, the needle comprising:
a proximal end for connection to a syringe; and
a distal end comprising a sharpened tip,
wherein the needle comprises a curved region between the proximal end and the distal end.

In an embodiment, the vertex angle of the curved region is between 130° and 179°.

In an embodiment, the needle comprises a first straight region extending from the proximal end to the curved region.

In an embodiment, the first straight region comprises a length of between 4 mm and 18 mm, optionally between 4 mm-12 mm, optionally between 4 mm-8 mm.

In an embodiment, the curved region terminates at the distal end.

In an embodiment, the vertex angle of the curved region is between 160° and 180°, optionally between 165° and 175°.

In an embodiment, the degree of bend of the curved region is between 5° and 50°, optionally between 10° and 45°, further optionally between 15° and 40°, and even further optionally between 20° and 35°.

In an embodiment, the curved region comprises an arc length of between 8 mm and 18 mm, optionally between 11 mm and 16 mm, optionally between 12 mm and 14 mm.

In an embodiment, the needle comprises a second straight region extending from the curved region to the distal end.

In an embodiment, second straight region comprises a length of between 7 mm and 12 mm, optionally between 8 mm-11 mm, optionally between 9 mm-10 mm.

In an embodiment, the vertex angle of the curved region is between 140° and 1600.

In an embodiment, the curved region comprises an arc length of between 2 mm and 5 mm, optionally between 3 mm-4 mm.

In an embodiment, the tip comprises an outer facing bevel.

In an embodiment, the tip comprises an inner facing bevel.

In an embodiment, the needle comprises an outer diameter of between 0.235 mm and 0.362 mm.

In an embodiment, the needle comprises an outer diameter of approximately 0.312 mm.

In an embodiment, the needle comprises a wall thickness in accordance with the ISO 6009:2016 wall standard definitions of regular-walled, thin-walled, extra thin-walled or ultra-thin walled.

In an embodiment, the needle comprises a wall thickness of less than 0.09 mm, optionally between 0.05 mm and 0.09 mm.

In an embodiment, the wall thickness of the needle tapers toward the distal end along at least a portion of the length of the needle.

In an embodiment, the needle is composed of a non-toxic, non-corrosive, durable, material.

In an embodiment, the material of the needle is selected from stainless steel, titanium, cobalt chrome and platinum.

In an embodiment, the needle is composed of stainless steel.

In an embodiment, the needle is between approximately 25-gauge and approximately 32-gauge. Optionally, the needle is approximately 30-gauge.

In an embodiment, the needle is packaged in a blister pack.

According to a second aspect of the invention, there is provided a method comprising injecting dermal filler into the dermal layer of skin via a needle according to a first embodiment of the invention.

In an embodiment, the method comprises injecting the dermal filler into the lip or perioral area of the face.

In an embodiment, the method comprises injecting the dermal filler into the lips wherein the needle is inserted at (or close to) the vermillion border aiming toward the body (wet/dry border) of the lip.

In an embodiment, method comprises retrograde depositing dermal filler as the needle is being at least partially withdrawn from the skin.

In an embodiment, the method comprises anterograde depositing dermal filler as the needle is being inserted further into the skin.

In an embodiment, the method comprises injecting a bolus deposit of dermal filler whilst the needle is static.

Throughout this specification and the claims which follow, unless the context requires otherwise:
the term “comprise” and variations thereof such as “comprises” and “comprising”, will be understood to include the inclusion of a stated integer or step or group of integers or steps but not the exclusion of any other integer or step or groups of integers or steps;
the terms “a” or “an” are intended to mean one or more; and
the terms “first”, “second”, “third” are used merely as labels, and are not intended to impose numerical requirements on, or to establish a certain ranking of importance, of their objects.

The present summary is provided only by way of example and not limitation. Other aspects of the present invention will be appreciated in view of the entirety of the present disclosure, including the entire text, claims, and accompanying figures.

### BRIEF DESCRIPTION OF THE FIGURES

FIG. 1 shows the layers of skin, including the epidermis, dermis and hypodermis.

FIG. 2 shows a needle according to the prior art injecting dermal filler into layers of the skin.

FIG. 3 shows components of a human lip.

FIG. 4 shows a needle according to the prior art injecting dermal filler into a human lip.

FIG. 5 shows a needle according to an embodiment of the present invention, injecting dermal filler into skin.

FIG. 6 shows a needle according to an embodiment of the present invention, injecting dermal filler into a human lip.

FIG. 7 shows the calculation of the curvature of the needle based on the arc length, chord length and vertex angle.

FIG. 8 shows various representations of needles according to embodiments of the present invention.

FIG. 9 shows various representations of a needle according to an embodiment of the present invention.

FIG. 10 shows various representations of a needle according to an embodiment of the present invention.

FIG. 11 shows the cross-sectional area of a needle having a ‘regular thickness’ wall, and a second needle having a ‘thin’ wall.

FIG. 12 shows photographs of two needles according to embodiments of the invention.

FIG. 13A shows human lips detailing tubercles, cupids bow and the vermillion border.

FIG. 13B shows human lips with straight line depositions of dermal filler using a straight needle.

FIG. 13C shows human lips with deposition of dermal filler using a straight needle along the cupid's bow.

FIG. 13D shows human lips with deposition of dermal filler in a straight line, using a straight needle, resulting in flattening out of the lower lip.

FIG. 14A shows human lips with deposition of dermal filler using a curved needle to facilitate a round bolus of dermal filler.

FIG. 14B shows human lips with deposition of dermal filler using a curved needle along the cupid's bow.

FIG. 14C shows human lips with deposition of dermal filler using a curved needle to provide a rounder, fuller deposition of dermal filler.

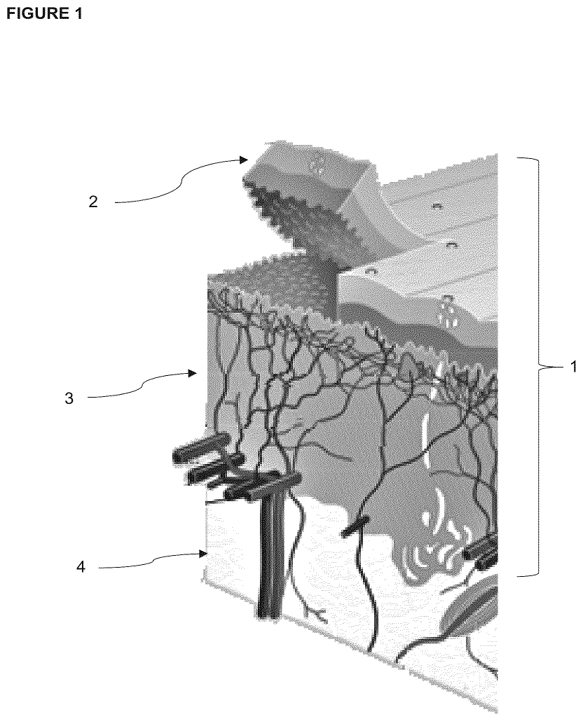

While the above-identified figures set forth one or more embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps, and/or components not specifically shown in the drawings.

### REFERENCE NUMERALS

1. skin
2. epidermis
3. dermis
4. hypodermis
5. needle
6. dermal filler
7. needle tip
8. submucosa
9. vermillion border
10. outer lip
11. sebaceous gland
12. muscle layer
13. salivary glands
14. wet/dry junction
15. chord length
16. vertex angle
17. arc length
18. first straight region
19. curved region
20. second straight region
21. inner diameter
22. outer diameter
23. tubercles
24. cupids bow
25. dermal filler depositions

### DETAILED DESCRIPTION OF THE INVENTION

In general, the invention relates to a curved needle for the injection of dermal fillers and other compounds into skin. In certain embodiments the needle is configured for injection of dermal fillers into the lips and perioral area of a human face.

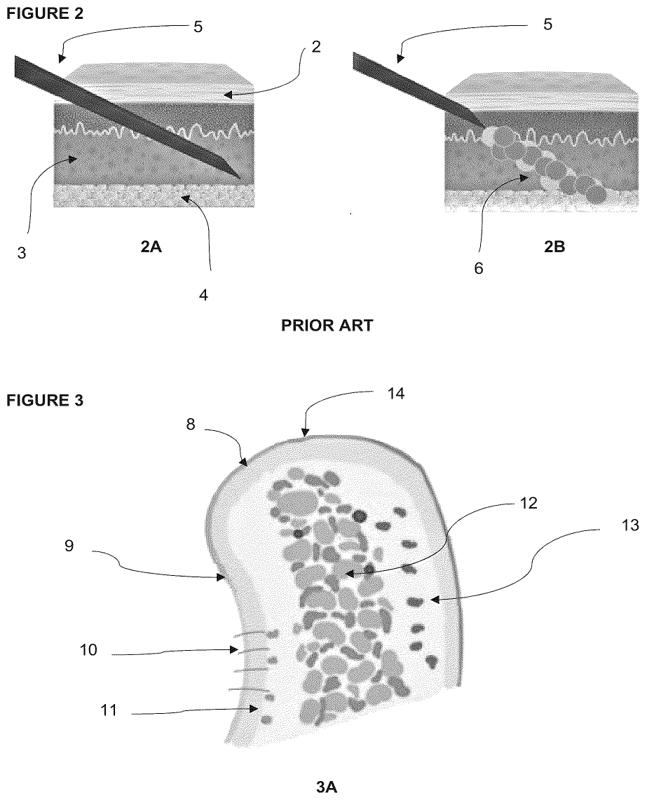

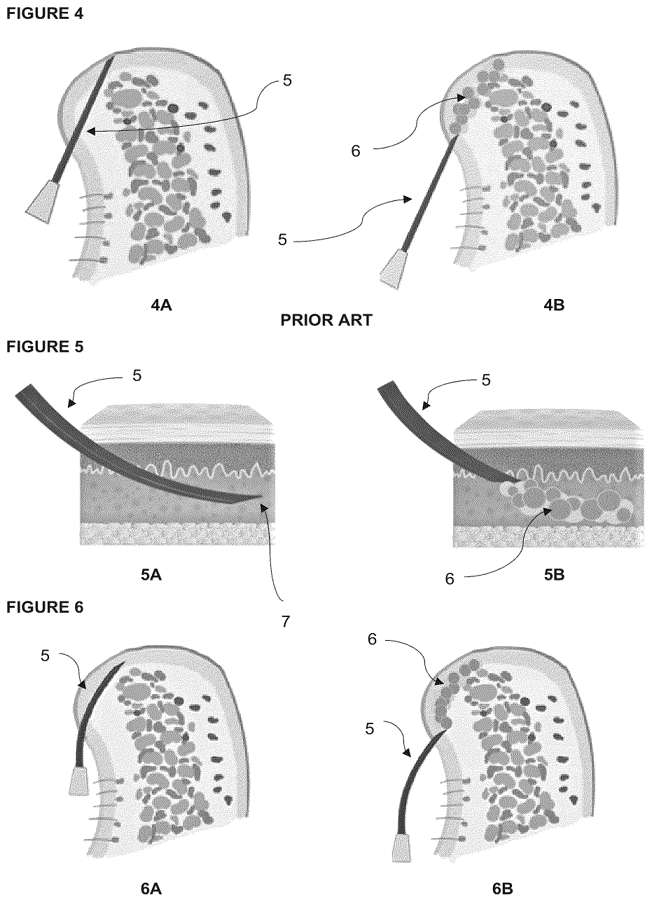

As previously discussed with reference to FIG. 2 and FIG. 4, it has been identified that use of a needle 5 according to the prior art can lead to injection of dermal filler 6 into undesired layers of skin 1, which risks unnecessary bruising, vascular occlusion and uneven cosmetic results.

FIGS. 3A and 3B show how a curved needle 5 according to embodiments of the invention may be used to reduce risks associated with needles of the prior art. As shown in FIG. 3A, it has been identified that use of a curved needle 5 (i.e. a needle comprising a curved region) may alter the angle at which the needle passes through and along skin layers, and improves the likelihood that a needle 5 will remain in a desired layer of skin 1, such as within the dermis 3. That is, with needles according to the prior art, the tip of the needle 7 will generally continue to follow the angle of entry into the epidermis 2, making it difficult to pass the needle 5 along through a layer of skin 1 without passing into another layer of skin. With a curved needle 5, it is possible to alter the angle of the needle tip 7 and distal end of the needle 5 more generally such that each may be passed along a particular layer of skin 1. As further shown in FIG. 3B, such a configuration is considered to improve the likelihood that dermal filler 6 will be injected into and remain in a desired layer of skin 1, thereby providing a desired cosmetic outcome.

FIGS. 3A and 3B demonstrate a retrograde method of depositing dermal filler 6, in which the needle 5 is first inserted to a desired position (see FIG. 3A), and dermal filler 6 is injected as the needle 5 is being (at least) partially withdrawn from the skin 1 (see FIG. 3B). In alternative embodiments of the present invention, dermal filler 6 may be deposited using an anterograde method, in which dermal filler 6 is injected as the needle 5 is inserted into the skin 1. Each of the retrograde method and the anterograde method are considered ‘linear threading’ or ‘tunnelling’ methods of deposition. An anterograde method may be commonly performed for tear troughs or the vermilion border because it allows the medical professional to better visualize the dermal filler tracking. A retrograde method may be commonly used to fill in deeper wrinkles (such as around the nasolabial folds and marionette lines) or to plump the lips. While a curved needle 5 according to embodiments of the invention may be particularly suited for linear threading methodologies as discussed above, such a needle 5 may also be used in ‘multiple injection’ methods, such as the depot method, or more generally where a bolus deposit of dermal filler 6 is injected while the needle 5 is static.

FIGS. 6A and 6B further show the utility of a curved needle 5 when injecting dermal filler 6 into the lips. As shown if FIG. 6A, it is believed that using a curved needle 5 according to embodiments of the invention enable the tip 7 of the needle 5 to follow the shape of the lip and pass along a desired skin layer, such as the dermis 3. As shown in FIG. 6B, it is considered that this will further enable dermal filler 6 to be injected into a desired layer of lip skin to provide a desirable deposition of dermal filler 6, which would in turn reduce the risk of bruising, vascular occlusion and poor cosmetic results.

In addition to the safety benefits referenced above, it has been surprisingly found that use of a curved needle according to embodiments of the present invention can provide improved cosmetic results by facilitating deposition of dermal filler in a curved line matching the features of the human face. As noted with respect to the prior art, use of straight needles facilitate the deposition of filler in a straight line, which in turn provides filling of features in a straight line on the face. This is in contrast to what is desired whereby dermal fillers are intended to provide fuller, rounder features such as rounder lips.

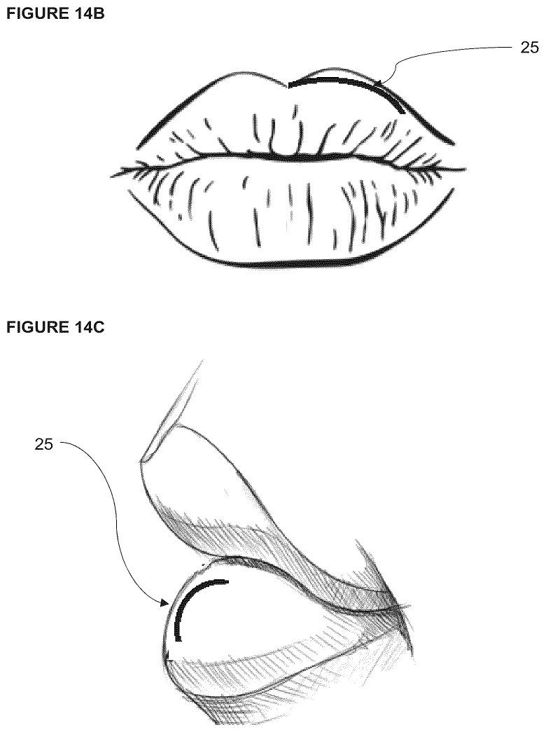

FIG. 14 shows various features of the face in which dermal filler is deposited in a curved line.

As shown in FIG. 14A, use of a curved needle according to embodiments of the invention facilitate a round bolus of dermal filler when filling the two lower tubercles 23. That is, to provide a round bolus using a curved needle according to the present invention, a practitioner may make a single insertion and provide for a first outer curved deposition following the natural path of the curved needle 5. Without removing the needle 5 altogether, the practitioner may retreat to an earlier position and then provide for further curved depositions, simply by following the natural path of the curved needle 5. FIG. 14A shows a round bolus produced from four curved deposition lines. The needle may be removed following completion of the four (in the example shown) deposition lines.

As shown in FIG. 14B, use of a curved needle 5 facilitates the practitioner following the cupid's bow when making a deposition of dermal filler 25. That is, the curved line of the cupid's bow 24 can be followed simply by allowing the path of the deposition 25 to follow the natural course of the curved needle 5.

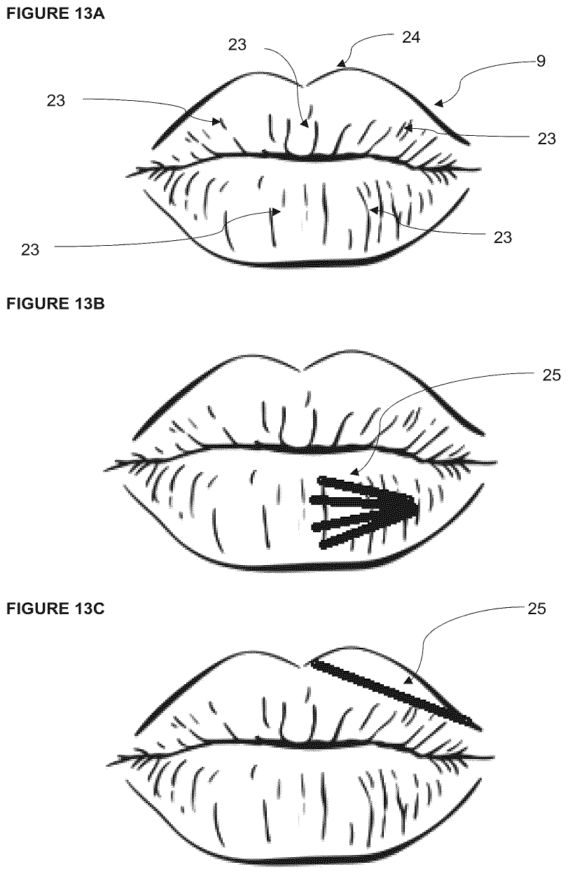

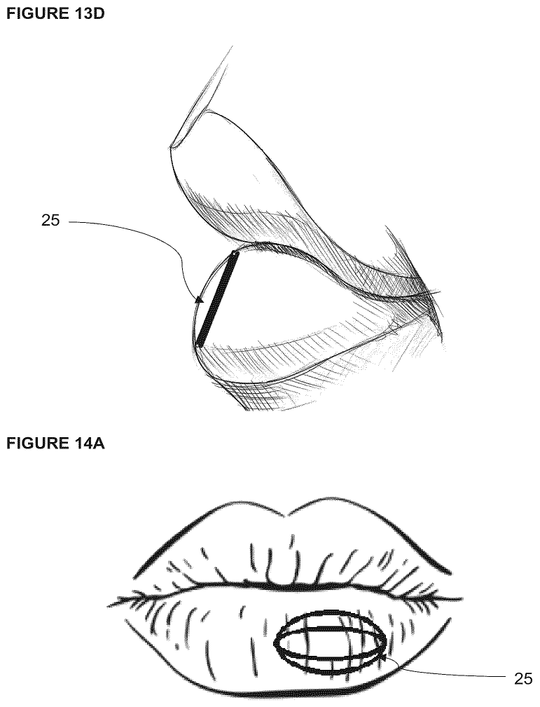

As shown in FIG. 14C, use of a curved needle 5 also facilitates the practitioner providing a rounder, fuller deposition of dermal filler 25. As noted earlier with respect to FIG. 13D, practitioners often make vertical injections into the lower lip to ‘fill out’ the lower lip to increase height and definition. Using an existing needle often results in deposition of dermal filler in a straight line, providing the opposite of the desired result whereby instead of providing a fuller, rounder effect—the deposition results in flattening out of the lower lip. In contrast to the prior art, use of a curved needle according to the invention facilitates the provision of a rounded deposition of filler 25 into the lower lip, enabling a fuller, rounder lower lip structure.

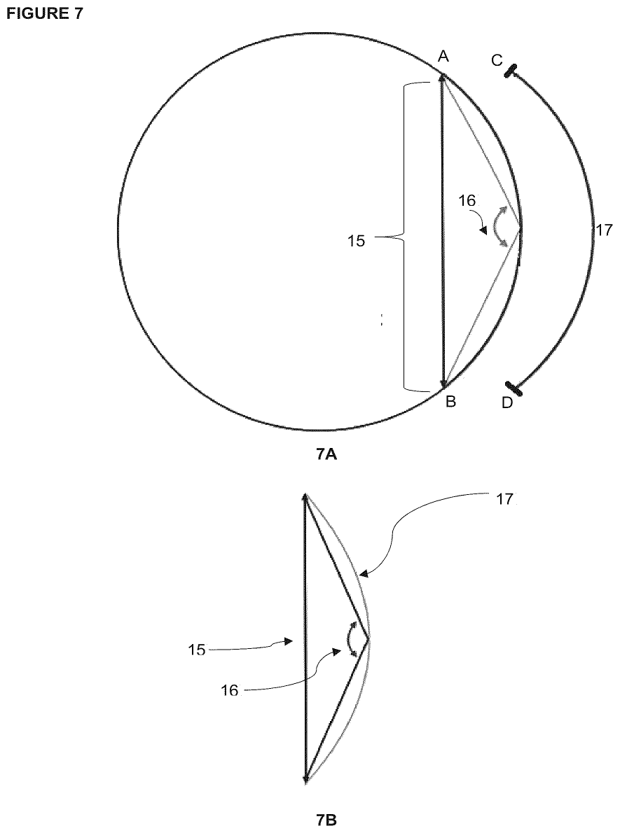

With reference to FIG. 7, a manner in which properties of the curvature of a curved region 19 of a needle 5 can be described is now explained. The curvature of a needle 5 may be seen as approximating a segment of the circumference of a circle whereby the curvature comprises:
an arc length 17, representing the distance along an arc between two end points, e.g. the distance along the curvature between points A and B, or the distance along the curvature between points C and D.
a chord length 15, representing the distance along a line segment whose end points intersect the end points along an arc.

A vertex angle 16, representing the vertex angle of an isosceles triangle which touches an arc at its endpoints (such that the entire chord length represents the hypotenuse of the triangle) and a point equidistant from the end points (such that the vertex angle touches the arc at this equidistant point). Throughout this specification and the claims that follow, the term ‘vertex angle’ is to be construed in accordance with this definition.

In describing the curvature of a needle by reference to terms such as ‘arc length’, ‘chord length’ and ‘vertex angle’, it is not intended that the curvature of the curved region is necessarily circular. Rather the curved region may adopt any generally curved shape such as a circular shape, an elliptical shape, a parabolic shape, a hyperbolic shape and for example any combination or combinations of the above. In this context and to the extent possible, the terms ‘arc length’, ‘chord length’, and ‘vertex angle’ are to be construed in the context of a curved region as more generally representing the length of the curved region following the curve, without requiring the curved region to be circular or any other specific curved shape.

An additional manner of determining the properties or extent of any curved region is by defining the degrees of bend, which simply relates to the extent of change in direction of the needle from the start of the curved region to the end of the curved region, measured in degrees such that:
a straight region having no curvature would have a degree of bend of 0°;
a curved region in which the direction of end of the curved region is at a right angle to the direction of the start of the curved region would have a degree of bend of 90°; and
a curved region in which the direction of end of the curved region is parallel but opposite to the direction of the start of the curved region would have a degree of bend of 180°.

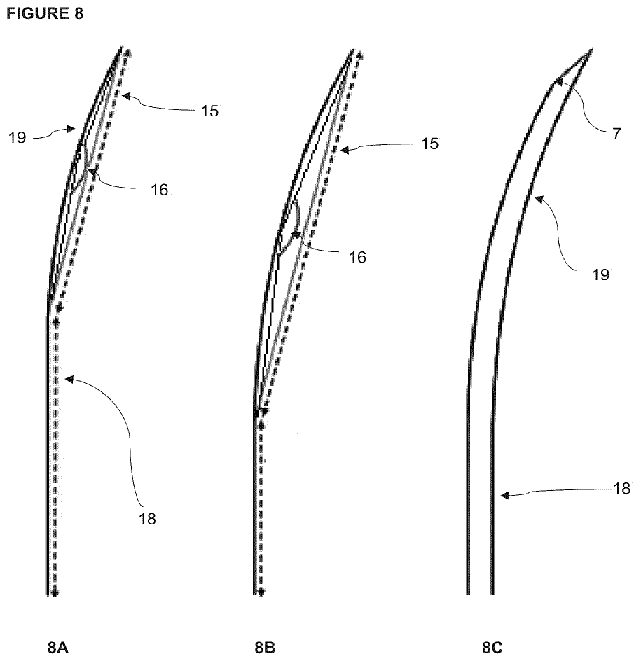

According to certain embodiments of the present invention, the curved region 19 of a needle 5 comprises:
an arc length 17 of between 8 mm and 18 mm, optionally between 11 mm and 16 mm, further optionally between 12 mm and 14 mm;
a vertex angle 16 of between 160° and 179°, optionally between 165° and 175°;
a chord length 15 of between 9-11 mm, and/or
a degree of bend of between 5° and 50°, optionally between 10° and 45°, further optionally between 15° and 40°, and even further optionally between 20° and 350.
   Such arrangements are further discussed with reference to FIG. 8.

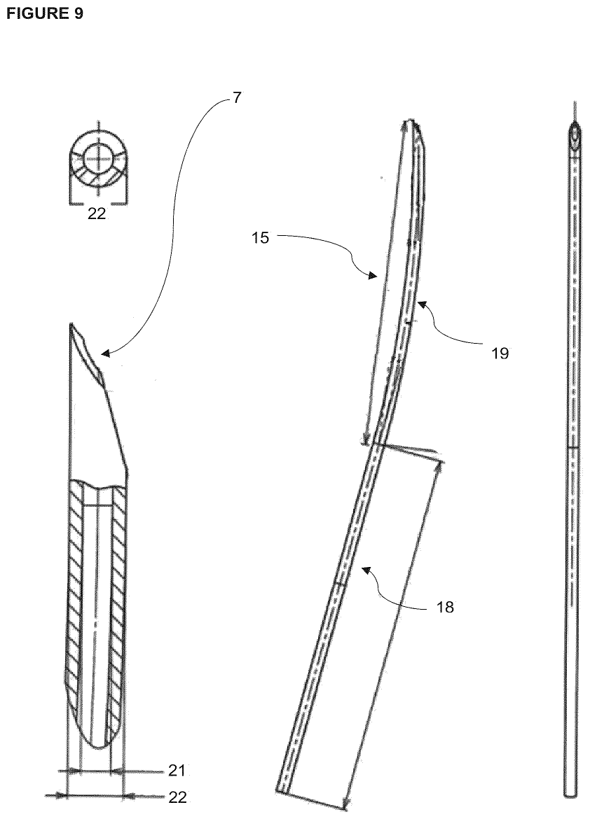

FIG. 8A, FIG. 8B and FIG. 8C each show needles according to embodiments of the present invention, each comprising a curved region 19 and a first straight region 18, whereby:
in FIG. 8A, the needle 5 comprises:
   a first straight region 18 that is 6.5 mm long; and
   a curved region 19 having a chord length 15 of 6.5 mm and a vertex angle 16 of 165° (and a degree of bend of approximately 35°);
in FIG. 8B, the needle 5 comprises:
   a first straight region 18 that is 4 mm long; and
   a curved region 19 having a chord length 15 of 9 mm, a vertex angle 16 of 165° and a degree of bend of approximately 35° (in other words, the needle 5 of FIG. 8A comprises a longer first straight region 18 and a shorter curved region 19 than that of FIG. 8A, notwithstanding that the degree of bend is approximately the same); and
in FIG. 8C, a needle 5 is shown having a needle tip 7 with an outward facing bevel. That is, according to certain embodiments of the invention, the tip 7 of the needle 5 is ‘bevelled’ or ‘angled’ to provide a sharper edge for insertion into layers of the skin 1. The bevelled tip 7 shown in FIG. 8C faces away from the curvature of the curved region 18, meaning that the tip 7 has an outward facing bevel. In alternative embodiments (as for example shown in FIG. 9), the tip 7 has an inward facing bevel whereby the bevel faces the direction of curvature of the curved region 19. In some embodiments, the needle 5 may include more than one bevel, such as by incorporating a double bevel design which (without wishing to be bound by theory) may provide for less painful injection.

FIG. 9 shows a needle 5 according to embodiments of the invention comprising a first straight region 18, a curved region 19 and a tip 7 comprising an outward facing bevel. The straight region 18 is approximately 10 mm long while the curved region 19 has a chord length 15 of approximately 9 mm, a vertex angle 16 of approximately 165° and a degree of bend of approximately 15°. FIG. 9 also shows the outer diameter and inner diameter of the needle 5, whereby the outer diameter 22 is approximately 0.309 mm and the inner diameter 21 is approximately 0.165 mm (providing a wall thickness of approximately 0.072 mm). This approximates a thin walled ‘30 gauge’ needle thickness profile. In referring to wall thickness and needle diameters, it is noted that processes of providing a curved region 19 may slightly alter the wall thickness and diameter of the needle within and in the vicinity of the curved region 19. For example, the process of providing a curved region 19 may reduce the diameter of the needle 5 in the curved region 19, or generally ‘flatten’ the cross-sectional shape of the needle 5 to provide a more elliptical cross-section. References to diameters, wall thicknesses and gauges are therefore to be construed as allowing for reasonable variability, particularly within and in the vicinity of the curved region 19, as a result of the production process.

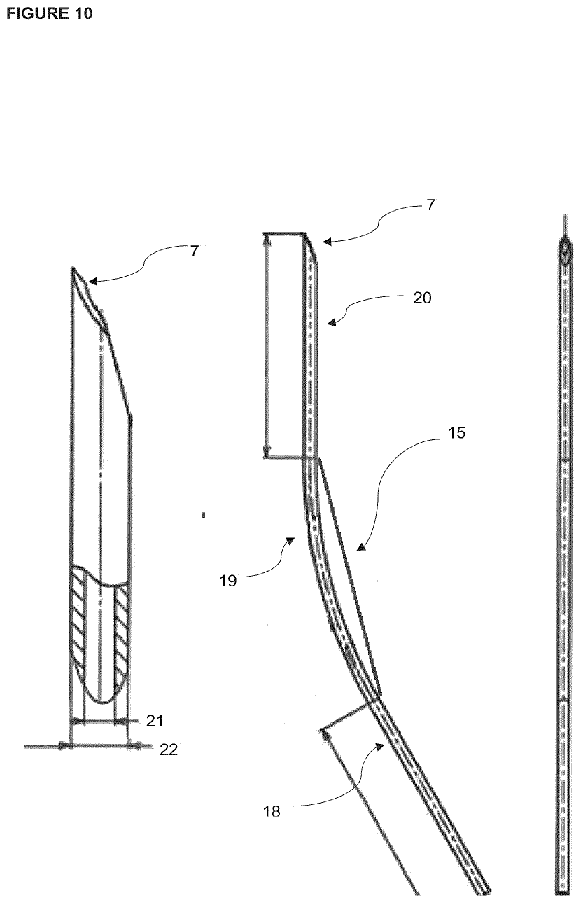

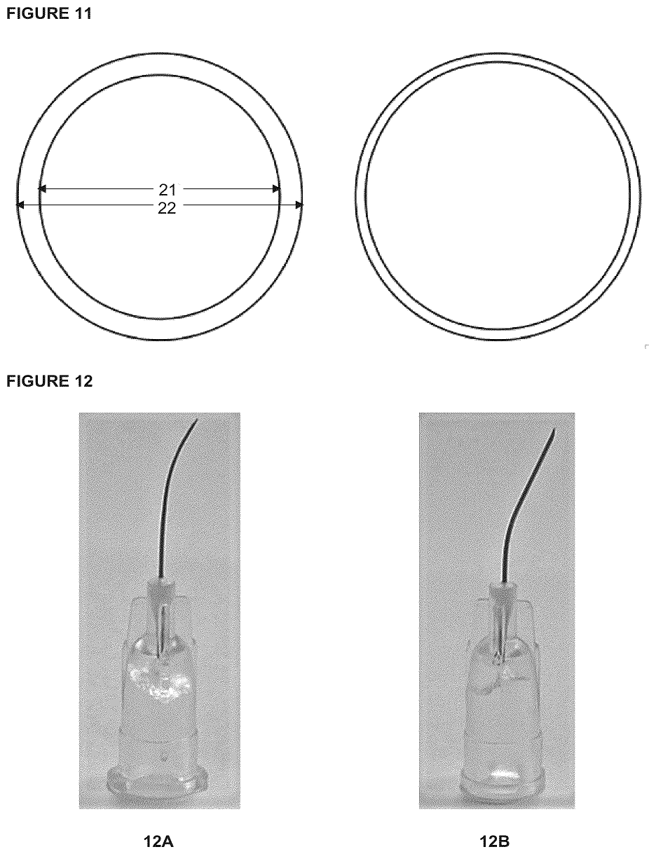

FIG. 10 shows a needle 5 according to embodiments of invention comprising a first straight region 18, a curved region 19, a second straight region 20 and a tip 7 comprising an inward facing bevel. The length of the first straight region 18 is approximately 6 mm, the length of the second straight region 20 is approximately 6 mm, the chord length 15 of the curved region 19 is approximately 4 mm, the vertex angle 16 of the curved region 19 is approximately 150° and the degree of bend is approximately 30°. Like FIG. 9, FIG. 10 also shows the outer diameter 22 and inner diameter 21 of the needle 5, whereby the outer diameter 22 is approximately 0.309 mm and the inner diameter 21 is approximately 0.165 mm (providing a wall thickness of approximately 0.072 mm). This approximates a thin walled ‘30 gauge’ needle thickness profile. In an alternative embodiment, a needle 5 may comprise a standard thickness of between 25 gauge to 32 gauge, or a thin walled thickness profile of between 25 gauge to 32 gauge. FIG. 11 contrasts a thin walled thickness profile against a standard thickness profile of a needle 5.

As may be demonstrated by FIG. 9 and FIG. 10 as discussed above, in certain embodiments of the invention the curved region 19 of a needle 5 comprising a second straight region 20 may have a shorter chord length 15 than may be found in a needle 5 not comprising a second straight region 20.

To ensure that the needle 5 may be used for injections into the human body, a needle 5 according to embodiments of the invention may be composed of a non-toxic, non-corrosive, durable material, such as: stainless steel, titanium, cobalt, chrome or platinum. In an embodiment, the needle 5 is composed of stainless steel. In a further embodiment, the needle 5 is composed of stainless steel 440, which enables the production of very sharp tips 7.

Needles 5 according to embodiments of the invention may be manufactured according to any suitable process as understood by the person skilled in the art. A common process for the manufacture of straight needles involves tube drawing, whereby a tube is drawn through progressively smaller dies to produce the straight needle and the end of the needle is bevelled to create a sharp pointed tip. According to certain embodiments a straight needle may be bent to provide for a curved region 19 using a tool similar to a compression bending tool as used for larger pipes and tubes in the context of plumbing and like professions. According to certain embodiments, the straight needle may be heated prior to bending to manage issues such as metal fatigue and the like which may affect the strength or other physical characteristics of the bent needle 5. In alternative embodiments, other methods of providing a bent needle 5 may be utilised applying principles of rotary draw bending, mandrel bending, compression bending, or roll bending for example. According to certain embodiments, methods of providing a curved region 19 which maintains the strength of the needle 5 and prevents or minimises any reduction of the diameter and wall thickness of the needle 5 in and around the curved region 19 are utilised.

In an embodiment, the proposed needle 5 will be packaged in a blister pack to provide a safe and convenient way to store the needle 5 due to the sharpness of the tip. In alternative embodiments, the needle may be packaged with a curved or flexible cap or sheath adapted to conform the shape of the needle 5. In alternative embodiments the needle 5 may be packaged in a self-contained box shape container with a screw off hub cover to allow for syringe attachment.

Embodiments of the invention are also directed to methods of injecting a dermal filler 6 using a needle 5 comprising a curved region 19 such as that described above. In particular embodiments, the present invention is directed to a method of injecting dermal filler 6 into the dermis layer 3 of the lips or perioral area of the face. According to certain of those embodiments, the needle 5 is inserted at or close to the vermillion border 9 aiming toward the body (wet/dry) border of the lip. Further, according to certain of those embodiments, dermal filler 6 is injected as the needle 5 is partially withdrawn from the skin 1. In an embodiment, dermal filler 6 is injected as the needle 5 is being inserted further into the skin 1, and/or a bolus deposit of dermal filler 6 is injected whilst the needle 5 is static.

FIG. 12 shows prototypes of the proposed needles 5, the needles 5 comprising a Luer lock located at the proximal end of the respective needles 5.

To those skilled in the art to which the invention relates, many changes in construction and widely differing embodiments and applications of the present invention will suggest themselves without departing from the scope of the present invention as defined in the appended claims. Further, the disclosed and described needle 5 may be suitable for other applications not limited to the injection of dermal fillers 6. As such, unless the context otherwise requires use of the disclosed needle 5 need not be limited to use in the injection of dermal fillers 6 and other uses are possible. The disclosures and the descriptions herein are purely illustrative and are not intended to be in any sense limiting.

1. A needle for injecting dermal filler into the dermis, the needle comprising:
a proximal end for connection to a syringe; and
a distal end comprising a sharpened tip,
wherein the needle comprises a curved region between the proximal end and the distal end.

2. The needle according to claim 1, a vertex angle of the curved region being between 130° and 179°.

3. The needle according to claim 1, further comprising a first straight region extending from the proximal end to the curved region.

4. The needle according to claim 3, wherein the first straight region comprises a length of between 4 mm and 18 mm.

5. The needle according to claim 1, wherein the curved region terminates at the distal end.

6. The needle according to claim 5, wherein a vertex angle of the curved region is between 160° and 179°.

7. The needle according to claim 5, wherein the curved region comprises an arc length of between 8 mm and 18 mm.

8. The needle according to claim 3, further comprising a second straight region extending from the curved region to the distal end.

9. The needle according to claim 8, wherein the second straight region comprises a length of between 7 mm and 12 mm.

10. The needle according to claim 8, wherein a vertex angle of the curved region is between 140° and 160°.

11. The A needle according to claim 8, wherein the curved region comprises an arc length of between 2 mm and 5 mm.

12. (canceled)

13. (canceled)

14. The needle according to claim 1, comprising an outer diameter of between 0.235 mm and 0.362 mm.

15. (canceled)

16. (canceled)

17. The needle according to claim 1, wherein a wall thickness of the needle tapers toward the distal end along at least a portion of a length of the needle.

18. (canceled)

19. The needle according to claim 1, wherein the material of the needle is selected from the group consisting of stainless steel, titanium, cobalt chrome, and platinum, and combinations thereof.

20. (canceled)

21. The needle according to claim 1, wherein the needle is between approximately 25-gauge and approximately 32-gauge.

22. (canceled)

23. The needle according to claim 1, wherein the needle is packaged in a blister pack.

24. A method of injecting dermal filler into the dermis, the method comprising:
injecting the dermal filler via the needle of claim 1.

25. The method according to claim 24, and further comprising:
injecting the dermal filler into a lip or perioral area of a face.

26. (canceled)

27. The method according to claim 24, the method further comprising:
retrograde depositing dermal filler as the needle is being at least partially withdrawn from the dermis.

28. (canceled)

29. The method according to claim 24, the method further comprising:
injecting a bolus deposit of dermal filler whilst the needle is static.
